(19) United States
(12) Patent Application Publication
(10) Pub. No.: US 2005/0154612 A1
(43) Pub. Date: Jul. 14, 2005

(54) Communication server for an instrument management system

(75) Inventors: Kathleen M. Smith, Erie, PA (US); Marie LaFrance, Concord, OH (US); Leslie M. Logue, Edinboro, PA (US)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 10/754,954

(22) Filed: Jan. 9, 2004

(51) Int. Cl. G06F 17/60

(52) U.S. Cl. 705/2

(57) ABSTRACT

A communication server for interfacing an antimicrobial treatment device with an instrument management system. The server allows an instrument tracking system to receive and request data from a wide variety of antimicrobial treatment devices.

### FIELD OF THE INVENTION

The present invention relates generally to a documentation system for tracking medical instruments, and more particularly to a communication server for interfacing an antimicrobial treatment device with an instrument management system.

### BACKGROUND OF THE INVENTION

Medical instruments (e.g., surgical instrument, bowls, basins, endoscopes, light cables, heart pacing cables, video cameras, microsurgical and eye instruments, ophthalmic lenses, internal and external defibrillator paddles and dilators) used by healthcare organization are typically subject to an instrument management cycle comprised of the following repeated steps: (1) instrument use, (2) instrument cleaning, (3) instrument storage, and (4) instrument sterilization. Moreover, the medical instruments may be also undergo periodic maintenance, repair, and replacement.

Healthcare organizations require quick, efficient and safe instrument reprocessing procedures. In order to improve patient safety and outcomes, there is a need to validate these reprocessing procedures. Accordingly, computer-based instrument tracking systems (or materials management systems) have been developed to facilitate the planning, control and documentation of antimicrobial treatment of medical instruments. An example of a typical instrument tracking system is the Sterile Processing Microsystem (SPM) by Materials Management Microsystems.

One drawback to existing instrument tracking systems is the inability to communicate directly with antimicrobial treatment devices (e.g., sterilizers and washers) from a wide variety of manufacturers. In some cases, hospital personnel must manually enter data into the instrument tracking system from the antimicrobial treatment devices.

The present invention addresses these and other drawbacks of prior art systems to provide a communication server for interfacing an antimicrobial treatment device with an instrument management system.

### SUMMARY OF THE INVENTION

In accordance with the present invention, there is provided an information management system for tracking instruments, comprising: (1) at least one instrument tracking client, each instrument tracking client installed on a respective general purpose computer; (2) at least one antimicrobial treatment device; and (3) a communication server interface in communication with the at least one instrument tracking client and the at least one antimicrobial treatment device, said communication server interface programmed to: (a) request data from the at least one antimicrobial treatment device, (b) transmit requests from at least one instrument tracking client, (c) receive data from the at least one antimicrobial treatment device, (d) receive requests from the at least one instrument tracking client, and (e) transmit data to the at least one instrument tracking client.

In accordance with another aspect of the present invention, there is provided a communication server interface in communication with at least one instrument tracking client and at least one antimicrobial treatment device, said communication server comprising: (a) means for requesting data from the at least one antimicrobial treatment device, (b) means for transmitting requests from at least one instrument tracking client, (c) means for receiving data from the at least one antimicrobial treatment device, (d) means for receiving requests from the at least one instrument tracking client, and (e) means for transmitting data to the at least one instrument tracking client.

An advantage of the present invention is the provision of a communication server that permits antimicrobial treatment devices from different vendors to communicate with an instrument tracking system.

Another advantage of the present invention is the provision of a communication server that facilitates communication with instrument tracking software from a wide variety of vendors.

Still another advantage of the present invention is the provision of a communication server that communicates using an industry standard communications protocol.

A still further another advantage of the present invention is the provision of a communication server that facilitates validation of instrument reprocessing procedures.

Still another advantage of the present invention is the provision of a communication server that facilitates quick identification of malfunctioning, or improperly configured, antimicrobial treatment devices.

Yet another advantage of the present invention is the provision of a communication server that facilitates paperless record keeping for sterilization and decontamination documentation.

These and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a block diagram of an instrument management system including a communication server, according to a preferred embodiment of the present invention;

FIG. 2 is a first flow diagram illustrating the flow of function calls between instrument tracking clients and the communication server; and

FIG. 3 is a second flow diagram illustrating the flow of function calls between instrument tracking clients and the communication server.

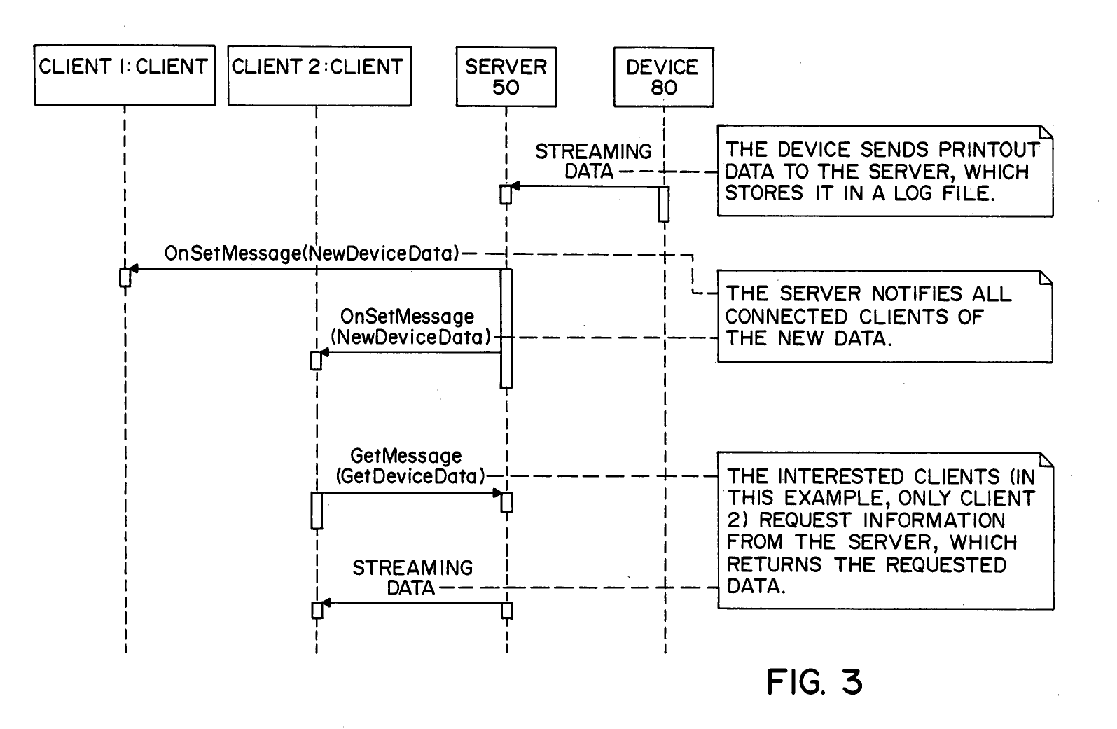

### DETAILED DESCRIPTION OF A PREFERRED EMBODIMENT

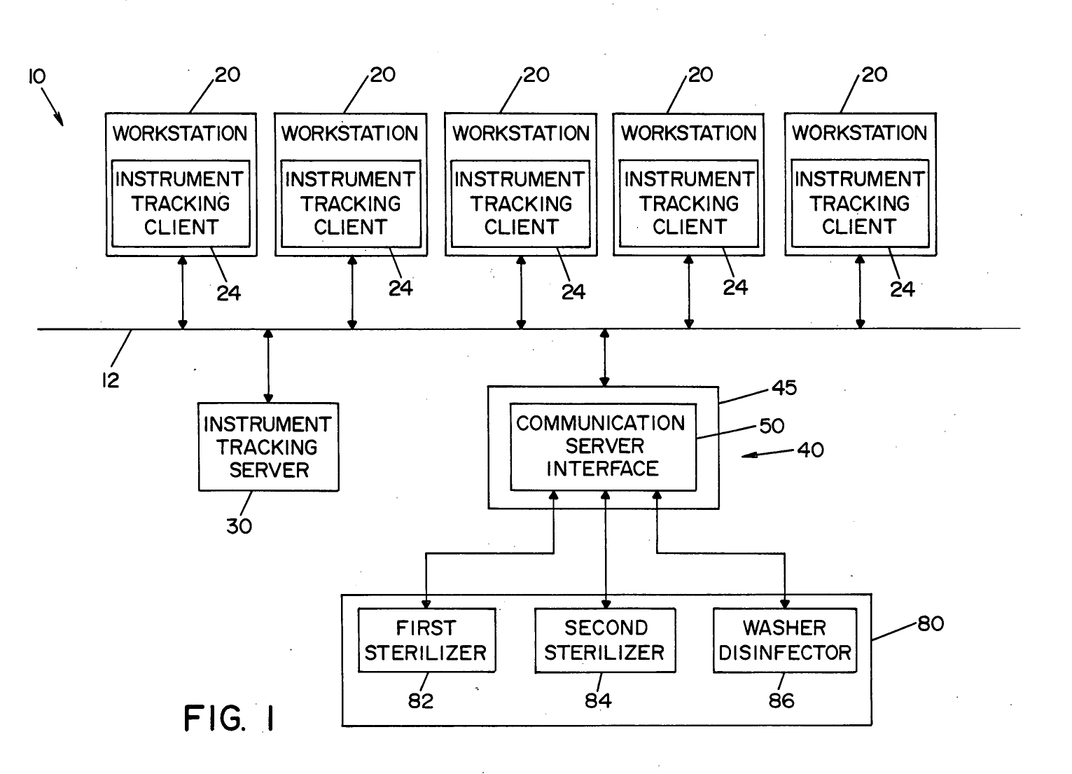

Referring now to the drawings wherein the showings are for the purpose of illustrating a preferred embodiment of the invention only and not for purposes of limiting same, FIG. 1 shows a block diagram of an instrument management system 10. Instrument management system 10 includes a plurality of workstations 20 with associated instrument tracking clients 24, an instrument tracking server 30, a communication server interface 40, and antimicrobial treatment devices 80. It should be appreciated that “antimicrobial treatment devices,” as used herein, includes, but is not limited to, devices for carrying out washing, sterilization, disinfection, decontamination, inactivation, and sanitization processes. The components of instrument management system 10 communicate via a communications network 12. Communications network 12 may include a private communications network, and/or a public communications network, such as the Internet.

Workstations 20 may take the form of a general purpose computer, such as a conventional personal computer (PC) system, including an input unit (e.g., keyboard, keypad, barcode scanner or touchscreen) and an output unit (e.g., a video or LCD display unit or printer). Workstations 20 may be physically located throughout a healthcare facility, as well as locations remote from the healthcare facility. For instance, workstations 20 may be located at: (1) a central services or central supply (CS) department (also known as central processing department (CPD)), where instruments are decontaminated, and prepared and packaged; (2) an operating room (OR); (3) a maintenance and repair facility; (4) administrative offices, and (5) accounting offices.

Instrument tracking server 30 is a network server computer that provides services to instrument tracking clients 24 on workstations 20. Instrument tracking server 30 and instrument tracking clients 24 comprise an instrument tracking system (ITS).

Antimicrobial treatment devices 80, include, but are not limited to a first sterilizer 82, a second sterilizer 84 and a washer disinfector 86. Antimicrobial treatment devices 80 may reduce microbial contamination by means of treatment agents (e.g., steam, ethylene oxide gas, peracetic acid and vaporous hydrogen peroxide), hot water, rinsing and drying.

Communication server interface 40 is comprised of a general purpose computer 45, such as conventional personal computer (PC), and a communication server 50. Communication server 50 is a server software program installed on computer 45 that serves instrument tracking clients 24. In this regard, communication server 50 sends requests for data, and fulfills requests for data, as will be described in detail below. Communication server 50 also operates to store data at computer 45. It should be understood that communication server 50 may alternatively be located on one of workstations 20, rather than computer 45.

In accordance with a preferred embodiment of the present invention, communication server 50 is implemented as a Component Object Model (COM), a COM+, and a Distributed Component Object Model (DCOM).

Component Object Model (COM) is a Microsoft® specification that defines the interaction between components in a Windows environment. A component is a self-contained coded module that provides some service to other components in an object-oriented environment. COM provides a set of interfaces allowing clients and servers to communicate within the same computer.

COM+ is both an object-oriented programming architecture and a set of operating system services. COM+ adds to COM additional system services for application components while they are running, such as notifying them of significant events or ensuring they are authorized to run. Among the services provided by COM+ are: (a) an event registry that allows components to publish the possibility of an event and other components to subscribe to be notified when the event takes place, (b) interception of designated system requests for the purpose of ensuring security, and (c) queues of asynchronously received requests for a service.

DCOM is the network version of COM that allows objects running in different computers attached to a network to interact. Accordingly, DCOM allows independent software components to communicate between different computers connected to the network. DCOM has several benefits, including, but not limited to: compatibility with multiple operating system platforms (e.g., Microsoft Windows, UNIX, Linux, Open VMS, and Solaris), compatibility with multiple software platforms (e.g., Visual Basic, C, C++, Java, Object Pascal, and COBOL), compatibility with multiple network protocols (e.g., TCP/IP, HTTP, and Novell), and allows implementation of security features to prevent unauthorized interfacing with the communication server software.

DCOM supports remoting objects by running on a protocol called the Object Remote Procedure Call (ORPC). The ORPC layer is built on top of distributed computing environment's Remote Procedure Call (RPC), and interacts with COM run-time services. A DCOM server is a body of code that is capable of serving up objects of a particular type at runtime. Each DCOM server object can support multiple interfaces each representing a different behavior of the object. A DCOM client calls into the exposed methods of a DCOM server by acquiring a pointer to one of the server object's interfaces. The client object then starts calling the server object's exposed methods through the acquired interface pointer, as if the server object resided in the client's address space.

Communication server 50 allows data from antimicrobial treatment devices 80 to be integrated into an instrument tracking client 24 (through the use of COM, COM+ and DCOM technology). In this regard, instrument tracking client 24 can directly monitor antimicrobial treatment devices 80 through communication server 50. For instance, instrument tracking client 24 can obtain cycle data including, but not limited to: (1) temperatures, (2) pressures, (3) cycle time, (4) operator name, (5) lot number associated with a specific load and operator name, (6) cycle start time, (7) total cycle time, and (8) cycle type.

Conventional instrument tracking systems may provide several functions, including, but not limited to: build and manage a master database of a healthcare facility's instrument inventory; track instrument sets; determine the correct type and quality of instruments and trays required allowing optimization of instrument set inventories; streamline or standardize instrument sets; locate needed instruments and trays; generate pick lists and service tickets; identify and track lost instruments and those needing repair or refurbishment; automation of sterilizer loads; manage staffing resources; improve instrument set turnaround time; create reports on inventory orders, repairs, needs projections, instrument set comparisons, productivity, sterilizer loads, and purchasing; facilitate staff training using online instrument and set images; track mobile equipment; and interface with OR scheduling software.

In accordance with a preferred embodiment of the present invention communication server 50 is compatible with a standard messaging protocol for clinical and administrative data. Preferably, the standard messaging protocol is HL7. The HL7 messaging standard promotes the exchange, management and integration of data that support clinical patient care and the management, delivery and evaluation of healthcare services.

Operation of instrument management system 10 will now be described in detail. Articles to be, or that have been, treated together using a given antimicrobial treatment process are referred to as a “Load.” A “Pack” is any pre-prepared instrument set, or alternatively may be a single instrument. A “Pack List” is created and maintained by the instrument tracking client 24. A “Cycle” refers to a defined sequence of operational steps of an antimicrobial treatment process. “Cycle Time” refers to the total elapsed time of a Cycle.

A “Load Number” is typically an integer that identifies an accumulation of Packs that have been placed together in an antimicrobial treatment device for a particular “Lot Number.” The Load Number is the sequential number of the Load. The Load Number increases by one each time a Cycle is executed. Load Numbers increase in this manner until midnight of each day, after which they reset to 01. “Open Load” refers to any Load that has not been processed, while a “Closed Load” refers to a processed Load. Pack information for a Closed Load is not typically edited or changed, but may be viewed for reports.

Sterilizer loads are tracked by automatic assignment of Lot Numbers by the instrument tracking client 24. For instance, the Lot Number may take the form of a 10-digit designation consisting of the date, an antimicrobial treatment device number, and a Load Number. As an example, Lot Number 083003-0201 refers to a date of Aug. 30, 2003, an antimicrobial treatment device number (02), and a Load Number (01). The Load Number increases by one each time a Cycle is selected. Load Numbers increase in this manner until midnight of each day, after which the Load Number resets to ‘01.’ An “Open Lot Number” refers to any Lot Number(s) assigned to Loads that have not been processed.

In accordance with a preferred embodiment, communication server 50 provides at least one of five different Lot Control modes, defined as follows:
(1) CPD with Operator: requires that the Load contents data be entered at the client workstation 20, and that the operator enter an operator identification (ID) code at the antimicrobial treatment device at the start and end of the cycle. A Lot Number is printed on a cycle tape printout.
(2) CPD without Operator: requires that the Load contents data be entered at a client workstation 20, but the operator code at the antimicrobial treatment device 80 is not required. A Lot Number is printed on a cycle tape printout.
(3) OR with Operator: does not require Load contents data be entered at a client workstation 20. The operator must enter an operator ID code at the sterilizer at the start and end of each cycle. A Lot Number is printed on a cycle tape printout.
(4) OR without Operator: does not require Load contents data to be entered at a client workstation 20, and does not require an operator ID code be entered at antimicrobial treatment device 80. A Lot Number is printed on a cycle tape printout.
(5) Non-Instrument Tracking Mode: does not require Load contents data entry or operator ID code entry. The Lot Number is not passed back and forth to antimicrobial treatment device 80, but the device 80 is still online as far as communication server 50 is concerned. Cycle tape information is gathered and may be displayed by client 24 on a status display screen.

It should be understood that load building and load management are functions managed by instrument tracking clients 24. By way of example, and not limitation, Loads for an antimicrobial treatment device 80 may be processed in the following manner in the “CPD with Operator” Lot Control Mode:
1) An antimicrobial treatment device 80 is selected and associated with a Load.
2) The operator logged into client 24 is associated with the Load.
3) A new Lot Number is created and assigned.
4) The Load is built by the operator by selecting from a list of item descriptions, set descriptions or packs.
5) The Load is closed. The operator verifies the contents of the Load matches the desired Lot Number, and verifies the Load is associated with the selected antimicrobial treatment device 80.
6) The Lot Number is sent to the selected antimicrobial treatment device 80.
7) The Load is placed in the selected antimicrobial treatment device 80.
8) The desired Lot Number is selected at the selected antimicrobial treatment device 80.
9) The desired cycle is selected.
10) The operator ID code is entered if in CPD with operator mode.
11) Client 24 parses “print and display data” from the selected antimicrobial treatment device 80 to determine the status of the Load (i.e., running or complete). The status information is sent to all clients 24, including Lot Number, Operator and a number associated with the selected antimicrobial treatment device 80. The status of the Load may be displayed at workstation 20.
12) Client 24 sends the Lot Number to the selected antimicrobial treatment device 80, where it is displayed with any others assigned to the same antimicrobial treatment device 80.
13) After the Load is processed by the selected antimicrobial treatment device 80, post cycle data may be added to the Load, such as the results of biological or chemical indicators.
14) The operator enters their operator code, and removes the Load from the antimicrobial treatment device 80.
15) Client 24 deletes the Lot Number from the antimicrobial treatment device 80.

By way of example, and not limitation, a typical instrument tracking client 24, may display: (1) current date; (2) all Loads for the current date; (3) state of the Load; (4) an empty status (i.e., the Load is created but contains no items); (5) a loading status; (6) a running status (i.e., Load being processed by an antimicrobial treatment device); (7) a complete status (i.e., antimicrobial treatment device 80 has finished processing the Load); (8) a canceled status; and (9) an indication of whether the Load contains a dart or a biological indicator.

It should be appreciated that antimicrobial treatment devices 80 may support tracking of treated articles by allowing a Physician ID, Patient ID or Procedure ID to be tied to a particular Load. Accordingly, server 50 supports the downloading of the of a Physicians list, Procedure ID or Patient ID.

In accordance with a preferred embodiment, communication server 50 supports the various Lot Control modes (described above), as well as various operations of clients 24, through the functions of SetMessage, GetMessage, OnSetMessage, and OnGetMessage. It should be appreciated that other communication protocols may also be implemented.

The SetMessage function allows any client 24 to issue a command to communication server 50. Communication server 50 then responds to the command, but does not return any data to client 24. In some cases, communication server 50 sends a command to an antimicrobial treatment device 80 in response to a SetMessage function.

The GetMessage function allows any client 24 to request data from communication server 50. Communication server 50 responds to the request by transmitting the requested data to client 24.

The OnSetMessage function allows communication server 50 to notify all clients 24 of updated information. Clients 24 determine whether the updated information pertains to it, and how to process the information.

The OnGetMessage function allows communication server 50 to request a client 24 to send information to communication server 50. Communication server 50 can then forward the information to an antimicrobial treatment device 80 that needs the information. Client 24 may send the requested information to communication server 50 using the SetMessage function.

In accordance with a preferred embodiment of the present invention, the SetMessage, GetMessage, OnSetMessage and OnGetMessage functions have an argument that is preferably an HL7 message. The HL7 message is a data string including a message name, field names, and field values.

As indicated above, the SetMessage function allows any client 24 to issue a command to communication server 50. Supported Messages and Fields for the SetMessage Function are listed and described in TABLE I.

TABLE I
SetMessage Function - Messages and Fields

| MessageName | Fields | Description |
|---|---|---|
| AddLotToDevice | LotNumber, | Client 24 sends this message either |
| | LotNumberFlag | by its own choosing or after receiving |
| | | an OnGetMessage. Server 50 |
| | | extracts the DeviceNumber from the |
| | | LotNumber and commands Device |
| | | 80 to add the LotNumber. Server 50 |
| | | may use the LotNumberFlag for |
| | | graphical user interface (GUI) display |
| | | purposes and to decide whether to |
| | | issue a regular or special ADD_LOT |
| | | command to the device. |
| AddOperatorToDevices | OperatorName, | Client 24 sends this message by its |
| | OperatorCode, | own choosing. Server 50 commands |
| | OperatorOffset, | all Devices 80 to add the operator. |
| | BarCodeNumber | |
| AddPhysicianToDevices | PhysicianName, | Client 24 sends this message by its |
| | Offset, | own choosing. Server 50 commands |
| | BarCodeNumber | all Devices 80 to add the physician. |
| DeleteLotFromDevice | LotNumber, | Client 24 sends this message by its |
| | LotNumberFlag | own choosing. Server 50 extracts |
| | | the DeviceNumber from the |
| | | LotNumber and commands the |
| | | Device 80 to delete the LotNumber. |
| DeleteOperatorFromDevices | OperatorOffset | The Client 24 sends this message by |
| | | its own choosing. Server 50 |
| | | commands all Devices 80 to delete |
| | | the operator. |
| LoadContentsChanged | LotNumber | The Client 24 sends this message by |
| | | its own choosing (when it detects the |
| | | change). Server 50 issues an |
| | | OnSetMessage to all other |
| | | connected Client 24 to notify them. If |
| | | they care about this LotNumber, they |
| | | can refresh their load information. |
| LoadStatusChanged | StatusIndicator | Client 24 sends this message by its |
| | | own choosing (when it detects the |
| | | change). Server 50 issues an |
| | | OnSetMessage to all other |
| | | connected Clients 24 to notify them. |
| SendControlCodeToDevice | DeviceNumber, | Client 24 sends this message by its |
| | ControlCode | own choosing. Server 50 sends the |
| | | ControlCode to the Device 80 as a |
| | | command. |
| SendPackInfoToDevice | DeviceNumber, | Client 24 sends this message either |
| | ItemDescription, | by its own choosing or after receiving |
| | PackCode | an OnGetMessage. Server 50 |
| | | commands the Device 80 to add the |
| | | ItemDescription and PackCode. |
| SystemConfiguration | FacilityName, | Client 24 sends this message by its |
| | NumberOfSterilizers, | own choosing. Server 50 updates its |
| | Field4, DateFlag, | registry with the information, as if a |
| | NumberOfBioReadings | user had entered it in the Configure |
| | | System form, and issues an |
| | | OnSetMessage to all other |
| | | connected Clients 24 to notify them. |

As discussed above, the GetMessage function allows any client 24 to obtain data from communication server 50. Supported Messages and Fields for the GetMessage function are listed and described in TABLE II.

TABLE II
GetMessage Function - Messages and Fields

| MessageName | Fields | Description |
|---|---|---|
| GetDeviceData | DisplayType, | After receiving a |
| | DeviceNumber | NewDeviceData |
| | | notification, any interested |
| | | Client 24 sends this |
| | | message. Server 50 |
| | | returns the requested data. |
| GetLotNumberFromDevice | DeviceNumber | A Client 24 sends this |
| | | message by its own |
| | | choosing. Server 50 |
| | | parses the Device 80 |
| | | display data to find the |
| | | LotNumber and returns it |
| | | to the Client 24. |

As indicated above, the OnSetMessage function allows communication server 50 to notify all clients 24 of updated information. Supported Messages and Fields for the OnSetMessage function are listed in TABLE III.

TABLE III
OnSetMessage Function - Messages and Fields

| MessageName | Fields |
|---|---|
| LoadContentsChanged | LotNumber |
| LoadStatusChanged | StatusIndicator |
| NewDeviceData | DisplayType, DeviceNumber, DisplayText |
| | (only when Display Type is zero) |
| DeviceConfiguration | DeviceNumber, DeviceName, |
| | DeviceModel, DeviceType, LotControl |
| SystemConfiguration | FacilityName, NumberOfSterilizers, |
| | Field4, DateFlag, NumberOfBioReadings |

As indicated above, OnGetMessage allows communication server 50 to request a Client 24 to send a message back to communication server 50. Communication server 50 forwards the information to Device 80. Supported Messages and Fields for the OnGetMessage function are listed and described in

TABLE IV
OnGetMessage Function - Messages and Fields

| MessageName | Fields |
|---|---|
| AddLotToDevice | DeviceNumber, LotNumberFlag |
| SendPackInfoToDevice | DeviceNumber, BarCodeNumber |

Set forth below is a summary of field names, including data type and a brief description.

| | Data | |
|---|---|---|
| Field Name | Type | Range and Definition |
| BarCodeNumber | | Text, usually digits, that is a value of a scanned barcode |
| ControlCode | Integer | 0-255 |
| DateFlag | Integer | One of the following integers: |
| | | 1 for USA: mm/dd/yy |
| | | 2 for ANSI: yy.mm.dd |
| | | 3 for Britain/France: dd/mm/yy |
| | | 4 for Germany: dd.mm.yy |
| | | 11 for Japan: yy/mm/dd |
| | | 12 for ISO: yymmdd |
| DeviceModel | Integer | For example, one of the following integers for the |
| | | corresponding devices 80: |
| | | 1 for Stage 3 |
| | | 2 for Stage 2 |
| | | 3 for Non 3000 |
| | | 4 for Century |
| | | 5 for System 2S |
| | | 6 for System 1 |
| | | 7 for Hoplab 130 |
| DeviceName | Char | The name of the Device 80 (e.g., VAC 01) |
| DeviceNumber | Integer | 1-30 |
| DeviceType | Integer | For example, one of the following integers: |
| | | 1 for Steam |
| | | 2 for Gas |
| | | 3 for 444 |
| | | 4 for 777 |
| | | 5 for 3400 |
| | | 6 for PIWS |
| | | 7 for 3000 SL |
| | | 8 for Floorloader |
| | | 9 for 3017 |
| | | 10 for Plasma |
| | | 11 for PAcid |
| | | 12 for Hoplab 130 |
| DisplayText | Char | Text appearing in the display screen associated with Device |
| | | 80 |
| DisplayType | Integer | 0 for Display data |
| | | 1 for Printout data |
| FacilityName | String | Name of healthcare facility |
| Field4 | Integer | 0 for Cost |
| | | 1 for Time (Labor Hours) |
| ItemDescription | String | Describes a Pack, (e.g., a heart pan set) |
| LotControl | Integer | 72 for Offline |
| | | 73 for OR without OPER |
| | | 74 for OR with OPER |
| | | 75 for CPD without OPER |
| | | 76 for CPD with OPER |
| LotNumber | Char | mmddyyXDDNN, where: |
| | | mm = two-digit month (01 = Jan, . . . , 12 = Dec) |
| | | dd = two-digit day (01, . . . , 28 or 29 or 30 or 31) |
| | | yy = two-digit year (03 = 2003, . . . , 99 = 2099) |
| | | X = normally a dash (-), but if it is in a cycle, it will be 1 |
| | | (for LEAK), 2 (for DART WARM), or 3 (for DART) |
| | | DD = two-digit DeviceNumber (01, . . . , 30) |
| | | NN = two-digit Load Number (01, . . . , 99) |
| LotNumberFlag | Integer | One of the following integers: |
| | | 0 for regular lot (to add to the list) |
| | | 1 for special lot (to issue real-time when the device |
| | | requests it) |
| MessageName | | For Server - SetMessage( ): |
| | | AddLotToDevice |
| | | AddOperatorToDevices |
| | | AddPhysicianToDevices |
| | | DeleteLotFromDevice |
| | | DeleteOperatorFromDevices |
| | | LoadContentsChanged |
| | | LoadStatusChanged |
| | | SendControlCodeToDevice |
| | | SendPackInfoToDevice |
| | | SystemConfiguration |
| | | For Server - GetMessage( ): |
| | | GetDeviceData |
| | | GetLotNumberFromDevice |
| | | For Client - OnSetMessage( ): |
| | | LoadContentsChanged |
| | | LoadStatusChanged |
| | | NewDeviceData |
| | | DeviceConfiguration |
| | | SystemConfiguration |
| | | For Client - OnGetMessage( ): |
| | | AddLotToDevice |
| | | SendPackInfoToDevice |
| NumberOfBioReadings | Integer | 1 or 2 |
| NumberOfSterilizers | Integer | 1-30 |
| OperatorCode | Char | Text |
| OperatorName | Char | Text |
| OperatorOffset | Integer | 0-9999 |
| PackCode | Char | Text |
| PhysicianName | Char | Text |
| PhysicianOffset | Integer | 0-9999 |
| StatusIndicator | | One of the following: |
| | | CLL<LotNumber> |
| | | to tell Clients 24 to close any open forms that are open for the |
| | | LotNumber |
| | | CST<LotNumber><OperatorName><DeviceNumber> |
| | | to tell the Clients 24 a cycle started (each field is fixed length, |
| | | space-padded to the right) |
| | | LDS |
| | | to tell the Clients 24 to refresh their device load control |
| | | displays |

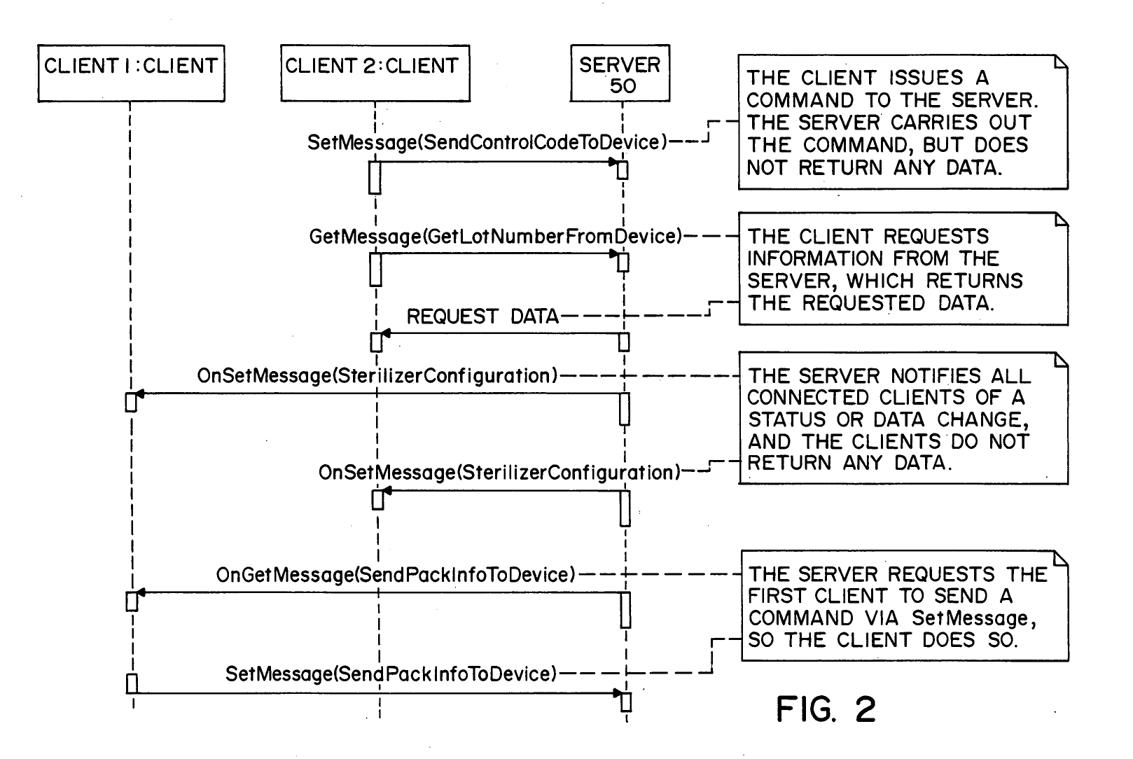

Referring now to FIGS. 1 and 2, there is shown a flow diagram illustrating examples for SetMessage, GetMessage, OnSetMessage and OnGetMessage functions. It should be understood that Client1 and Client2 refer to two different clients 24.

It is contemplated that antimicrobial treatment device specific messages may be developed and adopted as HL7 standard messages. These messages may include, but are not limited to:
(a) Sterilizer Command Message (SCM), a message sent by instrument tracking client 24 to program a sterilizer;
(b) Sterilizer Data Message (STD), a message sent from instrument tracking client 24 to obtain data from the sterilizer;
(c) Sterilizer Acknowledge Message (SAK), a message for the sterilizer to acknowledge receipt of a message;
(d) Sterilizer Notification Message (SNT), a message sent from the sterilizer to notify all instrument tracking clients 24 of updated information; and
(e) Sterilizer Request Message (SRQ), a message sent from the sterilizer to an instrument tracking client 24 to obtain data from the instrument tracking client 24.

Other modifications and alterations will occur to others upon their reading and understanding of the specification. It is intended that all such modifications and alterations be included insofar as they come within the scope of the invention as claimed or the equivalents thereof.

1. An information management system for tracking instruments, comprising:
at least one instrument tracking client, each instrument tracking client installed on a respective general purpose computer;
at least one antimicrobial treatment device; and
a communication server interface in communication with the at least one instrument tracking client and the at least one antimicrobial treatment device, said communication server interface programmed to:
request data from the at least one antimicrobial treatment device,
transmit requests from the at least one instrument tracking client,
receive data from the at least one antimicrobial treatment device,
receive requests from the at least one instrument tracking client, and
transmit data to the at least one instrument tracking client.

2. An information management system according to claim 1, wherein said communication server interface includes a communication server installed on a general purpose computer system.

3. An information management system according to claim 2, wherein said communication server is implemented as at least one of a Component Object Model (COM), a COM+, and a Distributed Component Object Model (DCOM).

4. An information management system according to claim 1, wherein said system further comprises a computer network for connecting said respective general purpose computer system with said communication server interface.

5. An information management system according to claim 1, wherein said antimicrobial treatment device is selected from the group consisting of: a sterilizer and a washer.

6. A communication server interface in communication with at least one instrument tracking client and at least one antimicrobial treatment device, said communication server comprising:
means for requesting data from the at least one antimicrobial treatment device,
means for transmitting requests from the at least one instrument tracking client,
means for receiving data from the at least one antimicrobial treatment device,
means for receiving requests from at least one instrument tracking client, and
means for transmitting data to the at least one instrument tracking client.

7. A communication server interface according to claim 6, wherein said communication server is installed on a general purpose computer system.

8. A communication server interface according to claim 6, wherein said communication server interface is connected with said at least one instrument tracking client and said at least one antimicrobial treatment device via a computer network.

9. A communication server interface according to claim 6, wherein said antimicrobial treatment device is selected from the group consisting of: a sterilizer and a washer.
